(19) United States
(12) Patent Application Publication
(10) Pub. No.: US 2022/0313863 A1
(43) Pub. Date: Oct. 6, 2022

(54) DEVICES AND SYSTEMS FOR CONCENTRATED BIOGENIC IONIZATION

(71) Applicant: John Chris Karamanos, Athens, TX (US)

(72) Inventor: John Chris Karamanos, Athens, TX (US)

(21) Appl. No.: 17/223,654

(22) Filed: Apr. 6, 2021

(51) Int. Cl. A61L 9/03; A61L 9/22

(52) U.S. Cl. CPC A61L 9/032; A61L 9/22; A61L 2209/11

(57) ABSTRACT

An improved biogenic ionizer with reduced acoustics and air entrainment is disclosed. The ionizer has a cation and anion generator connectable to a power source and utilizes needle point bipolar ionization to generate anions at a first set of electrodes and cations at a second set of electrodes. The electrodes are located on a first and second side of an air flow separator. The air flow separator is divided into a first air flow path way and a second airflow pathway which are at least partially separated from each other by a divider extending at least partially along the airflow separator. Discharged air through separated passages has a first air stream ionized substantially by cations and a second air stream ionized substantially with anions.

### TECHNICAL FIELD

This disclosure relates to devices and systems for biogenic ionization for improved Indoor Air Quality (IAQ) and Indoor Environmental Comfort (IEC) in various buildings and structures while maintaining a low Energy Use Intensity (EUI).

This disclosure further relates to devices and systems to remove aerosolized particulates, pathogens, pollens and contaminants such as viruses and pathogenic bacteria in a breathing area, such as for example, in a building, and reduce Sick Building Syndrome (SBS).

This disclosure further relates to devices and systems to utilize needlepoint bi-polar ionization to generate ions in an air stream to remove aerosolized particulates, pathogens, pollens and contaminants such as viruses and pathogenic bacteria in an enclosed building space and thereby reduce SBS.

This disclosure further relates to devices and system with improved air flow through to create ionized ions in the improved airflow through and remove aerosolized particulates, pathogens, pollens and contaminants such as viruses and pathogenic bacteria in a breathing area of a building or enclosed structure. The devices and systems thereby improve IAQ and IEC while maintaining or enhancing a building's EUI.

This disclosure further relates to devices and systems with improved airflow through to create a concentrated, charged and separated disbursements of positive and negative ions (cations and anions) in the improved airflow and direct predominantly the negative ions (anions) through a first side of the device and predominately positive ions (cations) through a second side of the device thereby enhancing the anion lifespan.

This disclosure further relates to devices and systems with improved airflow to create a concentrated, charged and separated disbursement of positive and negative ions in the improved airflow and flood a building breathe space or other internal area with ions and thereby avoid rapid deionization caused by the Coanda effect.

This disclosure further relates to devices and systems using variable geometry dampers to control the flow of ionized air through the device to provide for predominantly directional flow of positive ions (cations) through one side of the device and negative ions (anions) from another side of the device. The devices and systems permit an improved airflow of ionized air and flood a breathe space with ionized air and thereby avoid rapid deionization caused by Coanda effect.

This disclosure further relates to devices and systems using a unique “V” shaped air flow divider that separates the generation and flow of anions and cations from a bi-polar generator into separate airflows to improve life expectancy of the anions and cations and permit greater diffusion of ionized air through an air flow system. The system had self-cleaning ability to keep the electrode brushes clean in the cation and anion generators and improved spread of anions and cations through more efficient air flow.

### BACKGROUND

Devices for generating biogenic ionization for removing aerosolized pathogens, pollens, contaminants, and biologics in a closed space are known in the art. These devices generate an electric charge at a point or across a grid and draw or pass air over the electrified point or grid. Positive and negative ions (cations and anions) are randomly created in the process and the ions travel together in the airflow. Some ions interact with each other, whereas others contact a wall or other surface without interacting with a contaminant particle, pathogen or other biologic. Ionizing the air in a typical AHU loses its overall effectiveness by only cleaning a small portion of the air in the breathe area.

Ions have a life expectancy of 5-60 seconds with a limited life cycle. Ions bond to metal, insulation and other surfaces further losing their effectiveness unless it is directly on a room surface. When installed in an AHU, the ions travel across the coils, through the duct work metal turning vanes, the air terminal metal damper and then through an air diffuser which may not have the proper isothermal uniform distribution pattern needed for full area coverage. Other factors such as stack effect when heating, stratification and stagnation zones in the area due to air distribution design factors affect system efficiency. Past attempts to generate and disperse ions have not been entirely satisfactory for enhancing IAQ and IEC while reducing SBS. There is a continuing need for devices and systems that are portable, create a large number of anions and a rapid airflow into a breathe area without widespread deionization caused by the Coanda effect.

Prior corona ionization uses an electrical current to create bipolar ionized air. The Corona ionizer applies a high-voltage electrical current composed of a flow of negatively charged electrons, to a metal prong or needle. Electrostatic repulsion causes the electrons to detach from the prong or needle, attaching themselves to the molecules or nitrogen and oxygen in the air, forming negative ions, which are attracted the static charge in the work environment thus neutralizing it. These ions also attract certain types of molecules in the work environment like dust and other air particulates. These particulates cluster around the ion, weighing it down and forcing it to fall to the ground thereby cleaning the air.

Corona ionization can further be divided into AC and DC. AC or alternating current ionization uses one emitter to produce both positive and negative ions. This type of ionization is mainly used to protect components during assembly. DC direct current uses separate positive and negative power supplies that run simultaneously to create bipolar ions. DC ionizers are more efficient at producing ions and use lower operating currents, making them a better fit for cleanroom applications.

Cation and anion generators are known and generally include an oscillation signal generating circuit, a boost transformer and a high voltage rectifying circuit. An input end of the oscillation signal is connected to the power source, and a primary electrodes of the boost transformer, which in turn is connected to the oscillation signal output end of the oscillation generating circuit. The input end of the high voltage rectifying circuit is connected with a secondary electrode of the boost voltage. An output end of the high voltage rectifying circuit is respectively connected with a negative high voltage discharge and a positive high voltage discharge electrode. Cation and anion generators permit simultaneous generation of anions and cations

### BRIEF DESCRIPTION OF THE DRAWINGS

FIG. 1 is a front perspective view of one embodiment of an exemplary ion dispensing system;

FIG. 2 is a cut away partial view of an air flow passage with one embodiment of the air flow separator with electrodes or brushes in place;

FIG. 3 is top view of the air flow passage diverter showing its structure with a cation brush or electrode on one side and an anion electrode or brush on a second side of the air flow passage diverter;

FIG. 4 is a perspective view side view of the air flow passage diverter of FIG. 3 with a bi-polar ionization unit and electrodes or brushes in place;

FIG. 5 is a another perspective view of another embodiment of the air separator diverter with locations for a set of anion brushes and a set of cation brushes;

FIG. 6 is another perspective view of the air passage separator of FIG. 6;

FIG. 7 is a front view of an Air Handling Unit with numerous bi-polar ion generators at various locations along an air flow path

FIG. 8 is a view of the plate to cover the Air Handling Unit;

FIG. 9 is a rear view of the Air Handling Unit of FIG. 7;

FIG. 10 is a rear view of the ion dispensing system of FIG. 1 with the Air Handling Unit of FIGS. 7-9 attached.

### DETAILED DESCRIPTION

All figures and examples herein are intended to be non-limiting; they are mere exemplary iterations and/or illustrative embodiments of the claims appended to the end of this description. Modifications to specifically-described devices, systems, the order of steps in processes, etc., are contemplated. The dispensing devices, systems and methods are capable of other embodiments and of being practiced and carried out in various ways. Also, it is to be understood that the phraseology and terminology employed herein are for the purpose of the description and should not be regarded as limiting. Moreover, discussion of the related art throughout the specification should in no way be considered as an admission that such related art is widely known or forms part of common general knowledge in the field.

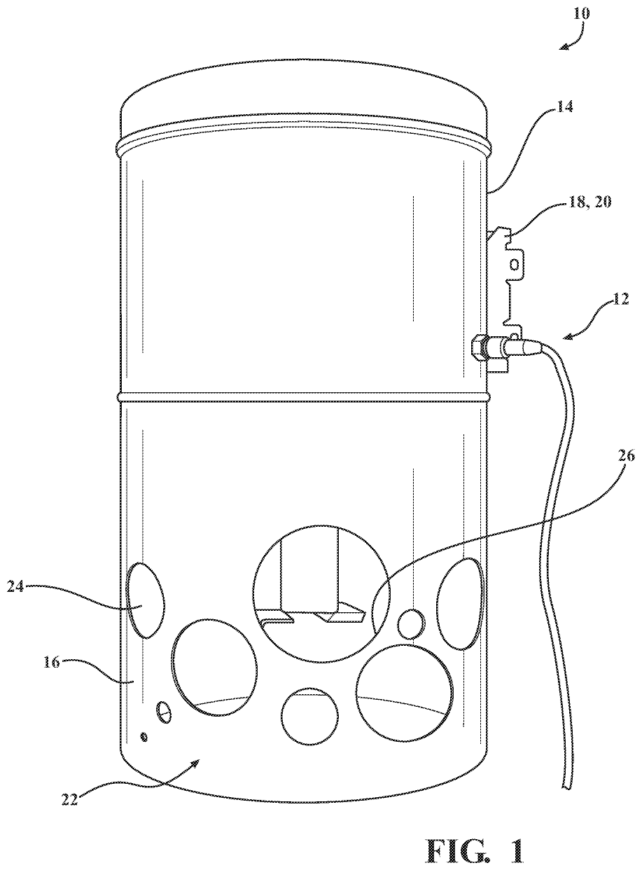

Turning now to the drawings wherein like numbers refer to like structures, FIG. 1 is a perspective front view of one embodiment of an ion dispensing device 10 having a body 12, defined between first end 14 and opposing second end 16. The first end has a first end mount 18 and the second end has a second end mount 20 the same as the first end mount whereby the device may be mounted in place. As shown, the body is cylindrical, but it is understood any shaped body may be desired. The device may be portable, or it may be affixed in place as the user may desire.

The second end of the body includes a vent portion 22, having variously shaped vents 24 to facilitate desired circulation of ionized air. The vents are shown as circular vents 26. Depending upon the airflow desired, it is contemplated the vents may be of any configuration as long as adequate air flow is maintained through the unit.

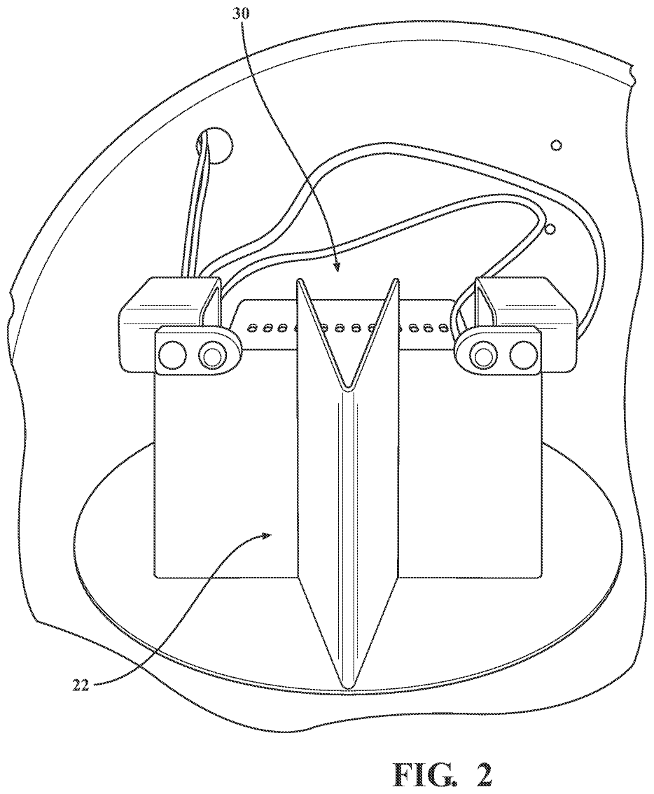

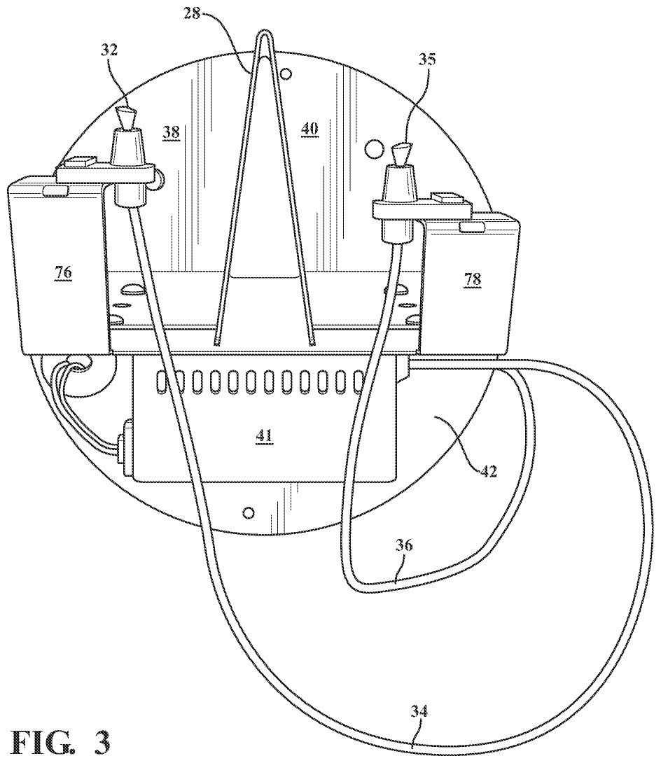

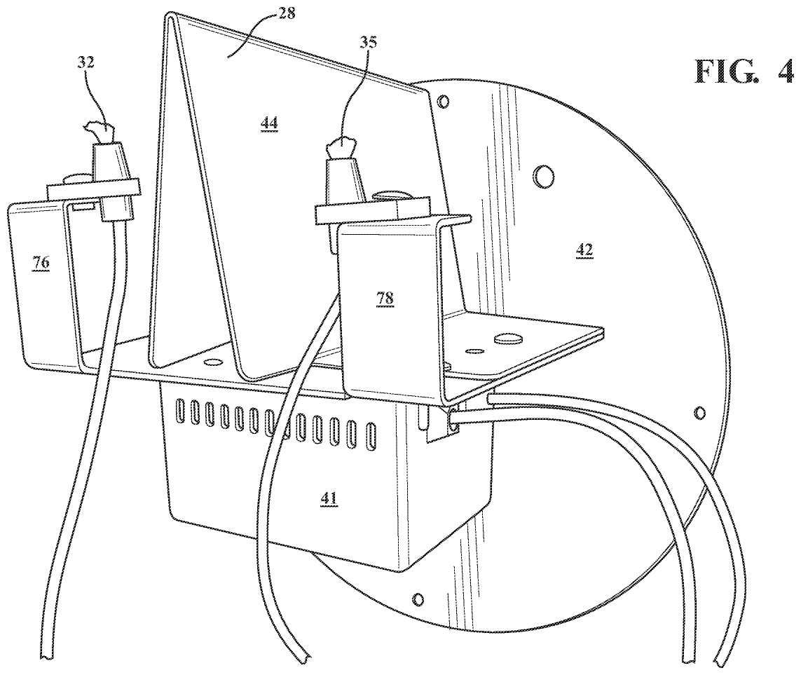

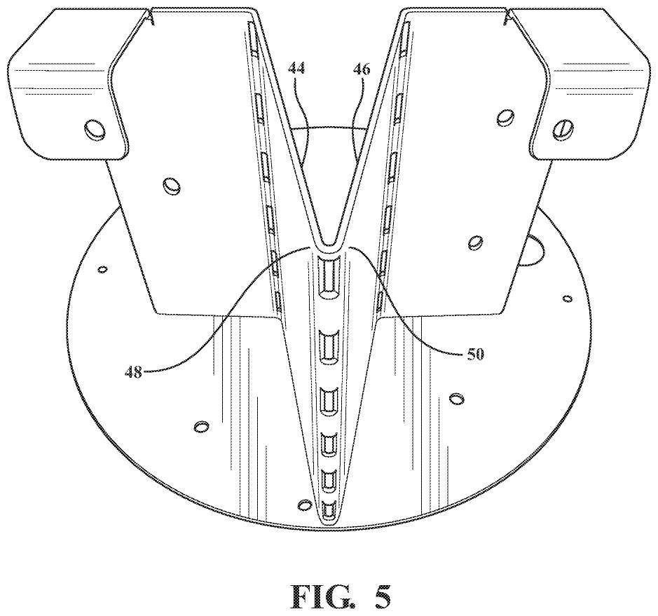

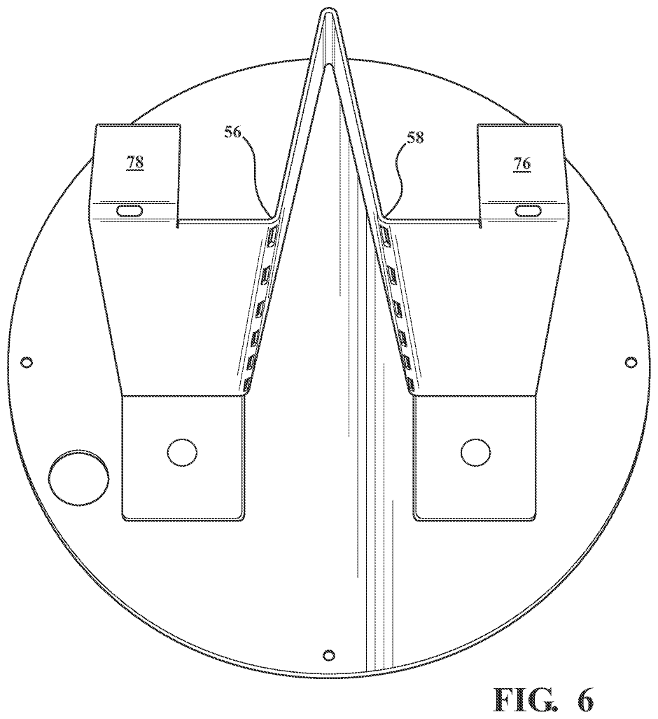

Turning to FIGS. 2-6, there is shown a V shaped air passage separator 28 in the air passage 30 of the unit to separate the flow of air into separate streams or flow. On a first side of the air passage, a first electrode or brush 32 is disposed or located and on the second side of the air passage a second electrode or brush is disposed or located. Each set of the electrodes or brushes is separated from the other set by the air passage divider which extends at least partially along the air passage. The electrodes or brushes are electrically connected at 34, 36 to the cation and anion bi polar generator. Anions are generated on the first side 38 and cations are generated at the second side 40. A bi-polar generator 41 is affixed to the unit. The Bi-polar generator may be any of the many on the market such as, for example, those available from Plasma Air of Hartford Conn. Those skilled in the art understand that the anions and cations may just as easily be generated on the opposite sides of the air passage separators than as shown, depending upon the cation anion generator wiring to the brushes. A stop plate 42 may be provided so the air passage separator may be mounted in the cylindrical body of the unit and direct air flow through the unit.

The air flow separator is a roughly “V” shaped article with sidewalls 44 and 46 oriented at acute angles relative to each other. While a V shape is shown, it is understood the separator could have walls oriented in a range of from about 5 degrees to 180 degrees relative to each other. Side wall 44 first end 48 is shown intersecting sidewall 46 first end 50 and forms an air passage 52 which is in fluid communication with the ambient air being moved through a variable or multi-speed fan in the device. Side wall 44 has a second end 54 and side wall 46 has a second end 56 that create an air flow outlet 58. A divider 62 extending at least partially there between defines a first air flow passage 64 and a second air flow passage 66. The air flow separator has a first side 68 and a second side 70 corresponding to the first and second airflow passages. Disposed in and along said first side is a first set of electrodes or brushes 72 which may be include one or more electrodes or brushes. As second set of electrodes or brushes 74, which may be one or more electrodes or brushes, is disposed in and along said second side of the air flow separator. As shown the brushes are located at first and second flanges 76 and 78, respectively. The cationic and anionic generator generates cations substantially along one side of the air flow separator and generates substantially anions on the other side air flow separator. As air is forced through the unit, the cations and anions move in separate air flow passages and streams so that the life of the ions is greatly extended as they do not immediately interact with each other while being generated. The air flow separator allows for maximizing ion output by separating the negative and positive brushes so the ions they generate do not bind, thereby maximizing the total ion count from the device. The air flow separator facilitates changes in the air entrainment pattern as the 5-180 degree V shaped separator throws out more air per the application.

The air flow separator with brushes may be positioned near the air flow openings of the device to further maximize ion output. This arrangement places the brushes at the maximum pressure point of the fan to generate the most efficient ion output.

In another embodiment, the unit may include a control module with integrated or remote sensor hub that senses Volatile Organic Compounds (VOCs) and/or CO2 levels, temperature, humidity, speakers, motion, sound, and lights such as LED configured and run by a user's phone application or through a base building system. The sensor may also be configured to modulate fan speed based upon sensed occupancy of the breathe space or in disinfect mode based upon the size of the breathe space or area. The device may further be configured to operate on voice recognition command. The sensors may also operate to permit the device to optimize ion output commensurate with the total breathe space diffusion in an isometric pattern.

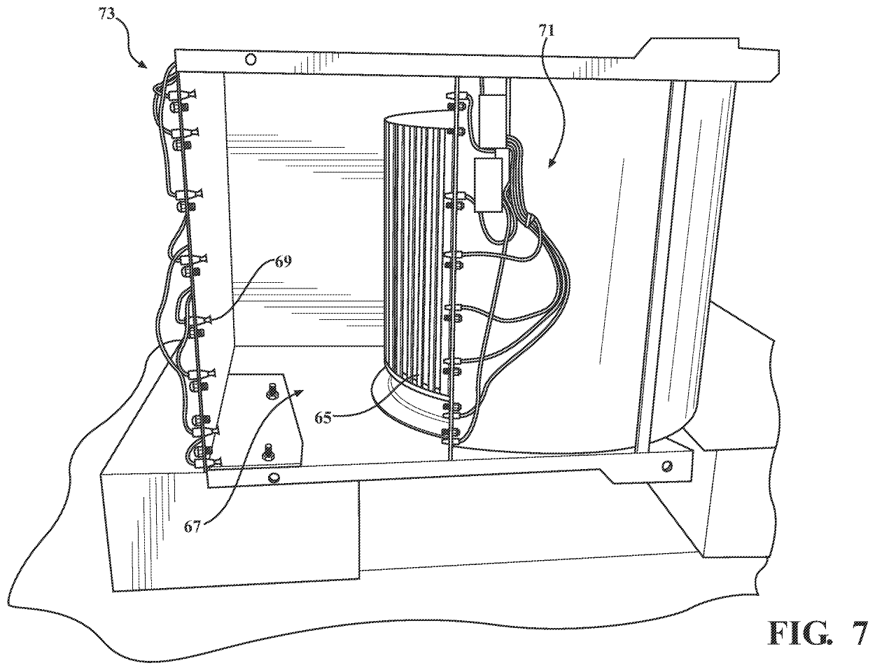

FIG. 7 shows a large AHU with various bipolar generators with brushes located in spaced apart relation to each other in the air passageway. Specifically, AHU 61 has a body 63 with a squirrel cage blower type fan 65 housed within. Air flow passage 67 has various brushes 69 in spaced apart relation to each other along opposed first and second sides 71, 73, respectively, of the AHU. Each brush is electrically connected to a bi-polar generator or biogenic ionizer, and the generators/ionizers electrically connectable to a power source. Cations are created at one side of the air passageway, and anions are created at the opposite side of the passageway. When the squirrel cage fan is activated, it ionized air through the AHU.

The brushes, regardless of invention embodiment described, may be cleaned by oscillating the brushes. In this regard, it is contemplated the brushes may be extended into or sheathed in a material, (metal or plastic or other suitable material) such as if in a drinking straw, so that as air blows across the brushes the oscillate to increase cleaning action and removal of debris. This may be accomplished by turning off the ion generator and increasing the fan speed to maximum CFM to cause the brushes to oscillate and self-clean. In another embodiment, it is contemplated to leave the ion generator operational and oscillate the brushes with the force of air blowing across them. In with case, the higher the air speed across the brushes, the more the brushes would oscillate.

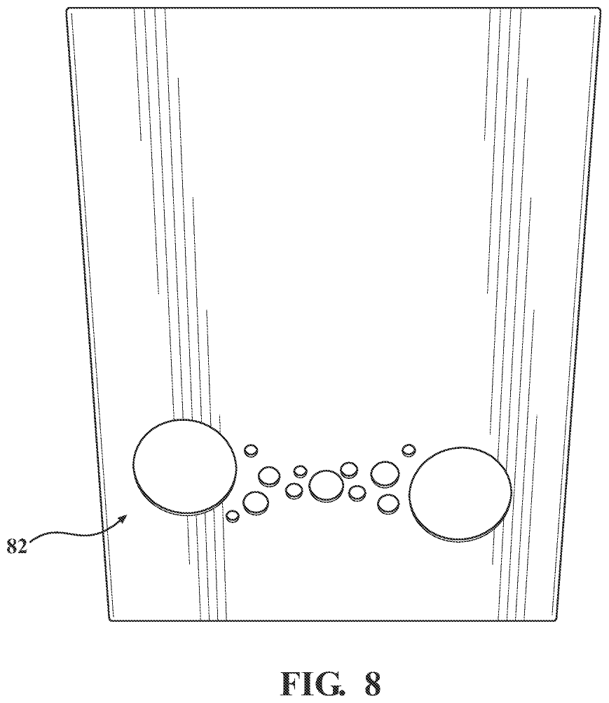

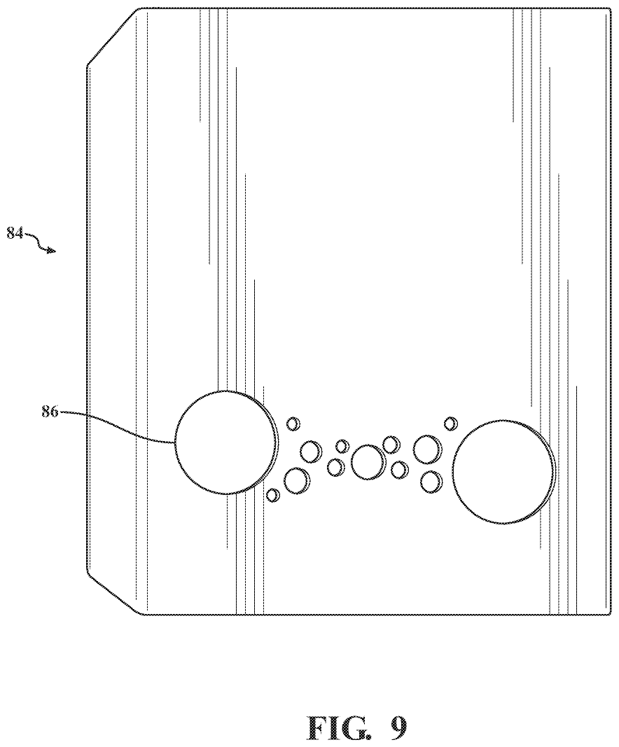

FIGS. 8-10 show one embodiment of an AHU that includes the biogenic ionizer of the present application. Plate 80 is a cover equipped with air inlet apertures 82. The plate, which may be removable, is a wall of the controller box 84. The box may include the biogenic ionizer as well as the air passages and dividers. The controller box is attachable to the body 10 of the device, and includes air outlet apertures 86 in its back side. The outlet aperture maybe positioned or fastened in fluid communication with apertures in the body 10 and air is drawn through the air inlet apertures 82 upon activation of the unit. Specifically, the biogenic ionizer is activated, and creates cation and anions on separate air flow passages as previously described. A fan in the body draws the ionized air through the unit and disburses it through vents 24.

The device as described may further be equipped with damper devices to vary the air flow/velocity though the openings 82. The damper may be a plate within the body such that rotation of the plate inside the unit progressively covers the outlet holes, thereby increasing velocity of air flow through the smaller holes. Rotating the plate in an opposite direct will increase the size of the opening, thereby increasing air flow through and decreasing air velocity.

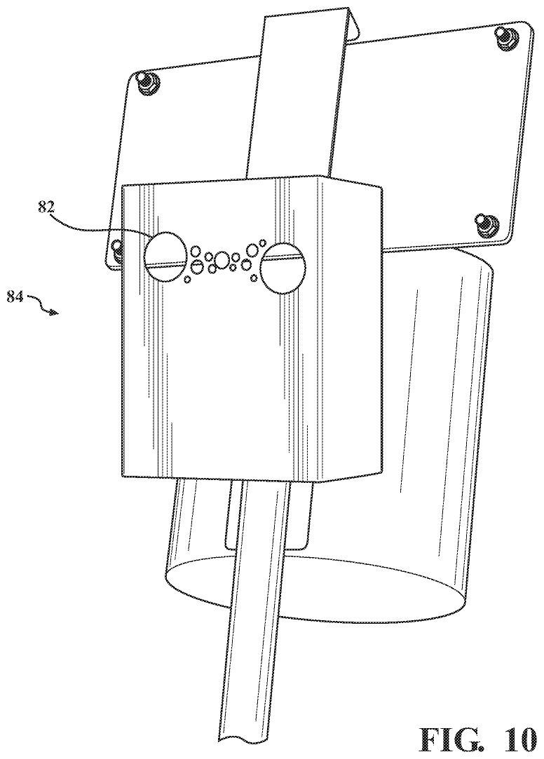

FIG. 10 shows the apparatus may be mounted on a pole 86 for positioning at any height in a closed space such as a room.

The device and systems as described may be used in a wide variety of applications and may be operated singly, in serial or in parallel with each other, depending upon the requirements of treating a breathe space or multiple breath spaces. The devices can be modulated such that it can be configured to a variety of breathe spaces. Different breath spaces require differing air flow to disinfect a given space. The actual cubic feet per minute (CFM) airflow may be determined for a given space and the operation of the device modulated to create that CFM. It is anticipated the device may be used in areas where as little as 125 CFM is required to as much as 2000 CFM is required to disinfect a given breathe space.

### EXAMPLES

### Example 1

A 600 square foot room was saturated with glycol vapor to simulate smoke contamination. An air meter was calibrated to ambient air conditions. The biogenic ionizer was located in close proximity to the ceiling of the room. The ionizer was activated and saturated the air in the 600 square foot room with 300,000 ion cc/minute at breathing zones for 17 minutes. The biogenic ionizer had a variable fan and moved the air through the biogenic ionizer to exit at about 1500 cfm. During the 17 minute period after start of ionization, the following was observed:
Glycol vapor was clearing by 5 minutes after start and was visibly cleared by 8 minutes after start of ionization.
Room air quality was restored to ambient outside air quality within 17 minutes of start of ionization.
An Ion meter in the room showed the ion count progressively dropped for 8 minutes after start of ionization as the glycol vapor was removed from the air. During the next 9 minutes after start of ionization, the ion count in the room air progressively increased, indicating the glycol vapor had been progressively ionized and removed from the room air. The ion count plateaued at 17 minutes after start of ionization at a level consistent with ion generation levels from the biogenic ionizer indicating the glycol vapor was substantially removed from the room air.

With regard to the processes described herein, it should be understood that, although the steps of such processes, have been described as occurring in a certain sequence, such processes could be practiced with the described steps performed in an order other than the exemplary order. It further should be understood that certain steps could be performed simultaneously, that other steps could be added, or that certain steps described herein could be omitted. In other words, the descriptions of processes herein are provided for the purpose of illustrating certain embodiments and should in no way be construed so as to limit the claimed invention.

Accordingly, it is to be understood that the above description is intended to be illustrative and not restrictive. Many embodiments and applications other than the examples provided would be apparent upon reading the above description. The scope of the invention should be determined with reference to the appended claims along with the full scope of equivalents to which such claims are entitled. It is anticipated and intended that future developments will occur, and that the disclosed systems and processes will be incorporated into such future embodiments. The invention is capable of modification and variation.

All terms used in the claims are intended to be given their broadest reasonable constructions and their ordinary meanings as understood by those knowledgeable in the technologies described herein unless an explicit indication to the contrary is made herein. Use of the singular articles such as “a,” “the,” “said,” recite one or more of the indicated elements unless a claim recites an explicit limitation to the contrary.

What is claimed as new and desired to be protected by Letters Patent of the United States is:

1. An improved biogenic ionizer with reduced acoustics and air entrainment, comprising; a cation and anion generator connectable to a power source; said cation and anion generator utilizing needle point bipolar ionization to generate anions at a first set of electrodes having at least one electrode and cations at a second set of electrodes having at least one electrode; said first set of electrodes located at a first side of an air flow separator and said second set of electrodes located at a second side of the air flow separator; said air flow separator having a first air flow path way at said first side and a second airflow pathway at said second side of said air flow separator, said first and second air flow pathways at least partially separated from each other by a divider extending at least partially along said airflow separator; said airflow separator having first and second outer side walls in proximal spaced apart location relative to each other at a first end to form an air flow inlet and extending away from each other toward a second end such that each second end of each of the outer side walls are in spaced apart relation to each other and form an air flow outlet larger than said air flow inlet; said first side of the airflow separator and the second side of the air flow separator defined by a divider extending at least partially between the outer walls; said first end of said air flow separator in fluid communication with a source of forced air and said second end of said air flow separator to discharge air that has been ionized during passage through said air flow separator; said discharged air having a first air stream ionized substantially by cations and a second air stream ionized substantially with anions.

2. The biogenic ionizer of claim 1, wherein set first set of electrodes includes more than one electrode.

3. The biogenic ionizer of claim 1, wherein said second set of electrodes includes more than one electrode.

4. The biogenic ionizer of claim 1, wherein said air flow separator is adjustable to facilitate air flow in an air pattern of from about 5 degrees to about 180 degrees.

5. The biogenic ionizer of claim 1, further including a fan to move ambient air through said air flow separator from the inlet to the outlet.

6. The biogenic ionizer of claim 5, wherein said fan is a mixed flow fan.

7. The biogenic ionizer of claim 1, further including a housing body having a length, width and height sufficient to accommodate said biogenic ionizer; said housing further including an air inlet and an air outlet in fluid communication with said air inlet in said air flow separator.

8. The biogenic ionizer of claim 7, wherein said housing body is cylindrical.

9. The biogenic ionizer of claim 1, wherein said electrodes may be cleaned by de-powering the cation and anion generator and increasing air flow velocity over the electrodes.

10. The biogenic ionizer of claim 1, wherein said electrodes are enclosed within a sheath and are cleaned of debris by increasing air flow over the brushes to oscillate the brushes.

11. The biogenic ionizer of claim 7, wherein the housing air outlet includes a damper to regulate the flow of air and air velocity.
